(19) United States
(12) Patent Application Publication
(10) Pub. No.: US 2026/0295158 A1
(43) Pub. Date: Oct. 1, 2026

(54) METHOD OF CONTROLLING PATIENT-CONTROLLED ANALGESIA

(71) Applicant: Too Jae Min, Seoul (KR)

(72) Inventor: Too Jae Min, Seoul (KR)

(21) Appl. No.: 19/091,097

(22) Filed: Mar. 26, 2025

(51) Int. Cl. A61M 5/172; A61M 5/14

(52) U.S. Cl. CPC A61M 5/1723; A61M 2005/1405; A61M 2205/3303; A61M 2205/3306; A61M 2205/3334; A61M 2230/62; A61M 2230/63

(57) ABSTRACT

The present disclosure relates to a method of controlling patient-controlled analgesia, performed by at least one processor of a computing device, the method may include predicting, based on test data obtained from a patient, whether the patient is in a hyperalgesia state, predicting, based on the test data, whether the patient is in an oversedation state, decreasing, when the patient is predicted to be in the oversedation state, a drug infusion amount of the patient-controlled analgesia by a set amount, and increasing, when the patient is predicted to be in the hyperalgesia state, the drug infusion amount of the patient-controlled analgesia by a set amount regardless of the oversedation state of the patient.

### TECHNICAL FIELD

The present disclosure relates to a method of controlling patient-controlled analgesia, and in particular, to predict a hyperalgesia state and an oversedation state based on test data obtained from a patient, and decrease, when the patient is predicted to be in the oversedation state, a drug infusion amount of the patient-controlled analgesia, and increase, when the patient is predicted to be in the hyperalgesia state, the drug infusion amount of the patient-controlled analgesia regardless of the oversedation state of the patient.

### BACKGROUND

Recently, when a patient develops persistent pain due to surgery or illness, a patient-controlled analgesia may be used to relieve the pain. The patient-controlled analgesia basically administers a certain amount of analgesic uniformly to the patient (which is referred to as “maintenance” in the present disclosure, and may be understood as a uniformly administered drug amount per hour.), when the patient feels particularly severe pain, they may press a button of the patient-controlled analgesia to temporarily administer more analgesic (this is treated as a separate drug administration from the aforementioned “maintenance.”). Nevertheless, in order to prevent misuse or overuse of analgesics, the button stops functioning for a certain period of time after the patient-controlled analgesia is pressed. However, this mechanism is dependent on the patient's manipulation, and thus may be less effective in situations where the patient's consciousness is dim. In addition, in a situation in which the parasympathetic nervous system of the patient is activated and is in an oversedation state, the pain felt by the patient is relatively small, and thus the infusion amount of the analgesic needs to be adjusted. However, such hyperalgesia state and oversedation state may be difficult for medical personnel to determine with the naked eye and may be less accurate for the patient to make his or her own determination. Therefore, there is a need for a method of flexibly adjusting the drug infusion amount of the patient-controlled analgesia of patient according to the hyperalgesia state and the oversedation state.

### SUMMARY

The technical problem to be solved by the present disclosure may be to predict a hyperalgesia state and an oversedation state based on test data obtained from a patient, and when the patient is predicted to be in the oversedation state, decrease a drug infusion amount of a patient-controlled analgesia, and when the patient is predicted to be in the hyperalgesia state, increase the drug infusion amount of the patient-controlled analgesia regardless of the oversedation state of the patient, thereby adjusting the drug infusion amount of the patient-controlled analgesia by considering when the patient is in the oversedation state and the hyper-pain state.

In addition, another technical problem to be solved by the present disclosure is to provide a rule-based method or an artificial intelligence model-based method for predicting the hyperalgesia state.

In addition, still another technical problem to be solved by the present disclosure is to provide a rule-based method or an artificial intelligence model-based method for predicting the oversedation state.

The technical problems to be achieved by the present disclosure are not limited to the above-mentioned technical problems, and still other technical problems not explicitly mentioned herein may be clearly understood by those skilled in the art from the description below.

To solve the aforementioned problem, relating to a method of controlling patient-controlled analgesia, performed by at least one processor of a computing device, the method includes predicting, based on test data obtained from a patient, whether the patient is in a hyperalgesia state, predicting, based on the test data, whether the patient is in an oversedation state, when the patient is predicted to be in the oversedation state, decreasing a drug infusion amount of the patient-controlled analgesia by a set amount, and when the patient is predicted to be in the hyperalgesia state, increasing the drug infusion amount of the patient-controlled analgesia by a set amount regardless of the oversedation state of the patient.

The present disclosure described above has the following effects.

By using the method according to an embodiment of the present disclosure, a drug infusion amount of a patient-controlled analgesia may be controlled in consideration of a hyperalgesia state and oversedation state of a patient.

In addition, by using the method according to an embodiment of the present disclosure, methods for predicting the hyperalgesia state of the patient may be provided.

In addition, by using the method according to an embodiment of the present disclosure, methods for predicting the oversedation state of the patient may be provided.

The effects of the present disclosure are not limited thereto, and various effects may further occur depending on the embodiment.

### BRIEF DESCRIPTION OF THE DRAWINGS

FIG. 1 is an illustrative diagram representing, by way of example, components of a computing device mentioned in the present disclosure.

FIG. 2 is an illustrative diagram representing an example for a method of controlling patient-controlled analgesia of a patient based on test data obtained from the patient, according to an embodiment of the present disclosure.

FIG. 3 is a flow chart showing an example of a schematic method of controlling patient-controlled analgesia of the patient, according to an embodiment of the present disclosure.

FIG. 4 is a flow chart showing a method of predicting a hyperalgesia state of the patient with a button operation signal of the patient, according to an embodiment of the present disclosure.

FIG. 5 is a flow chart showing a method of predicting the hyperalgesia state of the patient using a rule-based hyperalgesia prediction model, according to an embodiment of the present disclosure.

FIG. 6 is a flowchart showing a method of predicting the hyperalgesia state of the patient using an artificial intelligence hyperalgesia prediction model, according to an embodiment of the present disclosure.

FIG. 7 is a flowchart showing a method of predicting an oversedation state of the patient using a rule-based oversedation prediction model, according to an embodiment of the present disclosure.

FIG. 8 is a flowchart showing a method of predicting the oversedation state of the patient using an artificial intelligence oversedation prediction model, according to an embodiment of the present disclosure.

FIG. 9 is a flow chart representing a schematic flow for the method of controlling patient-controlled analgesia, according to an embodiment of the present disclosure.

Like reference symbols in the various drawings indicate like elements.

### DETAILED DESCRIPTION OF ILLUSTRATIVE EMBODIMENTS

Hereinafter, some embodiments of the present disclosure will be described in detail through exemplary drawings. In assigning reference numerals to the components of each drawing, it should be noted that the same components have the same numerals as possible, even if they are shown on different drawings. In addition, in describing the embodiments of the present disclosure, if it is determined that the specific description of the related known configuration or function interferes with the understanding of the embodiments of the present disclosure, the detailed description thereof will be omitted.

In addition, in describing the components of the embodiments of the present disclosure, terms such as first, second, A, B, (a), and (b) may be used. These terms are merely for distinguishing the components from other components, and the nature, sequence, order, or the like of the components is not limited by these terms. When a component is described as being “linked,” “coupled,” or “connected” to other component, it will be understood that the component may be directly linked or connected to the other component, but may also be “linked,” “coupled,” or “connected” through other component between each component.

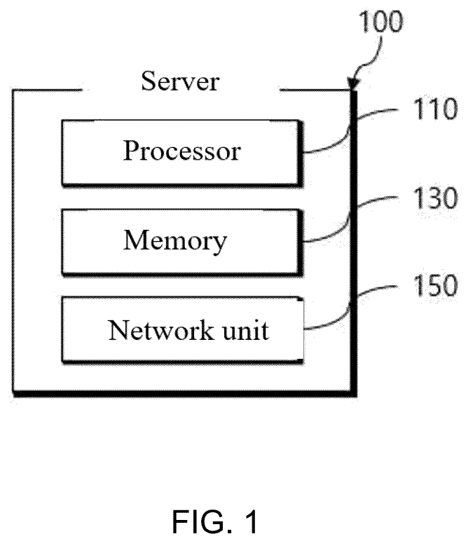

FIG. 1 is an illustrative diagram representing, by way of example, components of a server mentioned in the present disclosure. As shown in FIG. 1, computing device 100 may include a processor 110, a memory 130, and a network unit 150.

The processor 110 may be composed of one or more cores, and may include a central processing unit (CPU), a general purpose graphics processing unit (GPGPU), a tensor processing unit (TPU), and the like of the server. The processor 110 may predict the patient's oversedation state, predict the hyperalgesia state, and control a patient-controlled analgesia by executing instructions stored on the memory 130.

The network unit 150 may include a wired/wireless Internet module for network connectivity. The network unit 150 may perform communication with at least one of the nodes included in the blockchain network. As the wireless Internet technology, Wireless LAN (WLAN) (Wi-Fi), Wireless broadband (Wibro), World Interoperability for Microwave Access (Wimax), High Speed Downlink Packet Access (HSDPA), or the like may be used. As the wired Internet technology, Digital Subscriber Line (XDSL), Fibers to the home (FTTH), Power Line Communication (PLC), or the like may be used. The processor 110 may obtain test data obtained from the patient from the network unit 150. Conversely, the processor 110 may cause the network unit 150 to communicate instructions to the patient-controlled analgesia to control the patient-controlled analgesia.

The foregoing components are exemplary and the scope of the present disclosure is not limited to the foregoing components. That is, additional components may be included or some of the foregoing components may be omitted, depending on the implementation aspect for the embodiments of the present disclosure.

The computing device 100 may further include a memory 130. The memory may store a program for a step of the processor 110, and may also temporarily or permanently store input/output data. The memory may include at least one type of storage medium among a flash memory type, a hard disk type, a multimedia card micro type, a card-type memory (e.g., SD or XD memory, etc.), random access memory (RAM), static random access memory (SRAM), read-only memory (ROM), electrically erasable programmable read-only memory (EEPROM), programmable read-only memory (PROM), magnetic memory, magnetic disk, and optical disk. Such memory may operate under the control of the processor.

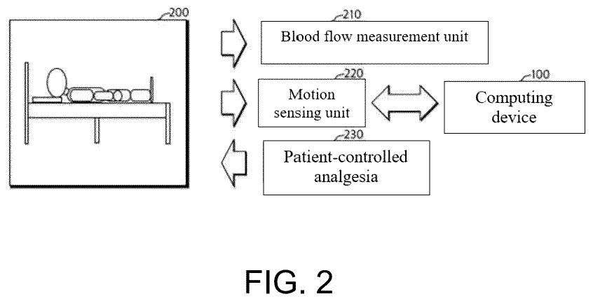

FIG. 2 is an illustrative diagram representing an example for a method of controlling patient-controlled analgesia of a patient based on test data obtained from the patient, according to an embodiment of the present disclosure.

Embodiments for controlling the patient-controlled analgesia of the present disclosure are now described. Referring to FIG. 2, paragraph number [0200] means a patient. The patient 200 is equipped with a blood flow measurement unit 210 and a motion sensing unit 220 as sensors. The patient 200 is also equipped with the patient-controlled analgesia 230 for relief of pain.

In this case, the blood flow measurement unit 210 and the motion sensing unit 220 may be instruments included in housings different from each other. However, the blood flow measurement unit 210 and motion sensing unit 220 may be sensors of wearable instrument housed together with a miniaturized computing device, such as a smartwatch. However, the blood flow measurement unit 210 and the motion sensing unit 220 are not limited thereto, and may exist in various forms.

More specifically, the blood flow measurement unit 210 may include a photoplethysmograph sensor. The photoplethysmography sensor may mean a technology of measuring the blood flow amount in peripheral blood vessels at a body part through which an artery passes, such as a fingertip of the patient, by using light. The blood flow amount in peripheral blood vessels changes according to the beating of the heart, and the processor 110 may calculate the heart rate or the oxygen saturation based on the measured blood flow amount.

Alternatively, the blood flow measurement unit 210 may be an instrument that measures blood flow with an amount of blood flow obtained by inserting needle into the blood vessel of the patient. However, it may be said that the above-mentioned photoplethysmograph method is more preferable in terms of measuring the pain relief of the patient and the movement of the patient.

The motion sensing unit 220 may include an acceleration sensor. The acceleration sensor may output a sensor value that is proportional to the acceleration of moving by itself. In addition, the motion sensing unit 220 may include a gyro sensor. The gyro sensor may output a sensing value related to whether it is currently positioned at a certain angle by itself. The gyro sensor values may be associated with tilt, roll, and yaw, each associated with an inclination direction.

The patient-controlled analgesia referred to in the present disclosure is called Patient Controlled Analgesia (PCA), and when the patient develops persistent pain due to surgery or illness, the patient-controlled analgesia may continuously infuse a certain amount of analgesic to the patient. The patient-controlled analgesia basically infuses a certain amount of analgesic uniformly to the patient, when the patient feels particularly severe pain, they may press a button of the patient-controlled analgesia to temporarily administer more analgesic. Nevertheless, in order to prevent misuse or overuse of analgesics, the button stops functioning for a certain period of time after the patient-controlled analgesia is pressed. The patient-controlled analgesia may include a Bluetooth module. The patient-controlled analgesia may communicate with the network unit 150 of the computing device 100 based on the Bluetooth module. For example, the processor 110 may transmit a signal regarding the drug infusion amount to the patient-controlled analgesia through the Bluetooth module of the patient-controlled analgesia. At this time, the drug infusion amount may be increased, decreased, or maintained, and the increase amount and the decrease amount may be adjusted by the processor 110.

The test data (sensing data) output from the blood flow measurement unit and the motion sensing unit may be transmitted to the computing device 100. Then, the processor 110 of the computer device 100 may predict whether the patient is in a hyperalgesia state and an oversedation state based on the test data, and control the drug infusion amount of the patient-controlled analgesia according to the predicted state.

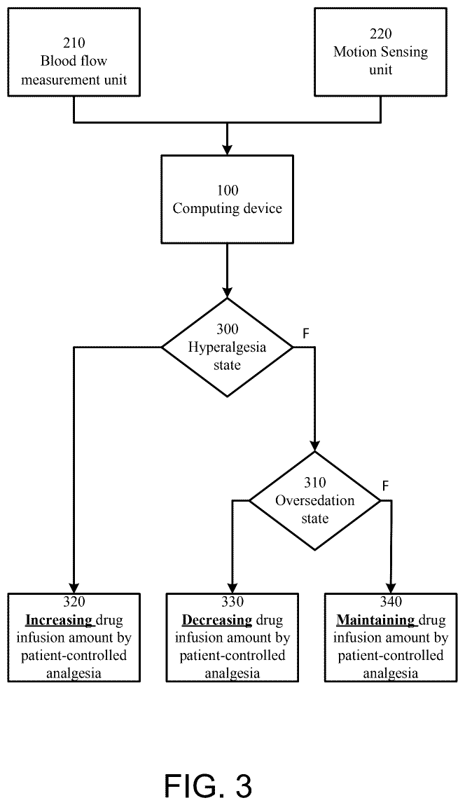

FIG. 3 is a flow chart showing an example of a schematic method of controlling patient-controlled analgesia of the patient, according to an embodiment of the present disclosure.

First, the processor 110 of the computer device 100 may obtain the blood flow amount, which is a type of test data of patient, from the blood flow measurement unit 210, and obtain a gyro sensor value and an acceleration sensor value, which are types of test data of the patient, from the motion sensing unit 220. Then, referring to paragraph number [0300], the processor 110 of the computing device 100 may predict (A) whether the patient is in the hyperalgesia state, and referring to paragraph number [0310], predict, (B) if the patient is not in the hyperalgesia state, whether the patient is in the oversedation state. At this time, as previously mentioned regarding (A) and (B), the processor 110 may subsequently perform (B) when it is determined that the hyperalgesia state predicted in (A) is negative, but the processor 110 may also perform (A) and (B) simultaneously. Nevertheless, from the viewpoint of saving processing resources, it may be said that the former case is more preferable.

Referring to paragraph number [0320], the processor 110 may increase the drug infusion amount of the patient-controlled analgesia if the patient is in the hyperalgesia state. Referring also to paragraph number [0330], the processor 110 may decrease the drug infusion amount of the patient-controlled analgesia if the patient is in the oversedation state. Of course, referring to the paragraph number [0340], the drug infusion amount of the patient-controlled analgesia may be maintained if the patient is not in both the hyperalgesia state and the oversedation state. Nevertheless, if the state of the patient is the hyperalgesia state, the processor 110 may cause the patient-controlled analgesia to increase the drug infusion amount regardless of the oversedation state of the patient.

Meanwhile, predicting the hyperalgesia state by the processor 110, as in the paragraph number [0300] or predicting the oversedation state, as in the paragraph number [0310], may be performed based on a rule-based model or an artificial intelligence model based on a predetermined policy. Details regarding this will be described later.

Meanwhile, alternatively, causing the patient-controlled analgesia to increase the drug infusion amount may pose a risk to the patient, depending on the action and side effects of the analgesic. Thus, the processor 110 may ignore the step of paragraph number [0300] and only perform the oversedation state prediction of paragraph number [0310]. As a result, the patient-controlled analgesia may cause a decrease in the drug infusion amount 330, or the patient-controlled analgesia may cause the drug infusion amount to be maintained 340.

Additionally, the processor 110 of the present disclosure may classify the hyperalgesia state and the oversedation state without using sensor values predicted by the blood flow measurement unit. At this time, in classifying the hyperalgesia state and the oversedation state, the processor 110 may utilize the movement amount, which may be calculated based on the sensor values of the motion sensing unit, as a basis for classification.

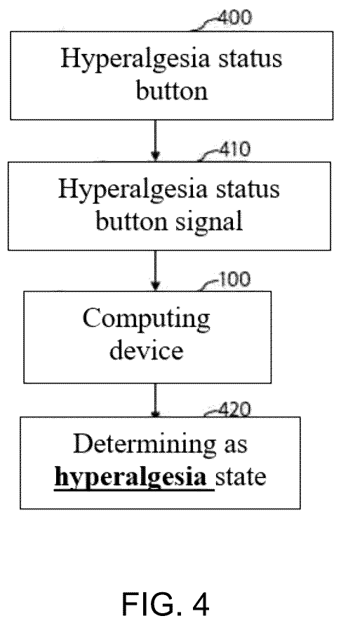

FIG. 4 is a flowchart illustrating a method of predicting the hyperalgesia state of a patient with a button operation signal of the patient according to an embodiment of the present disclosure.

As previously mentioned, the patient-controlled analgesia may include the hyperalgesia status button that the patient may manually manipulate. The hyperalgesia status button 400 may generate an hyperalgesia status button signal 410, which is an electrical signal, to cause the computing device 100 to recognize that the hyperalgesia status button is activated. If the processor 110 recognizes the hyperalgesia status button signal, the processor 110 may determine the patient to be hyperalgesia regardless of the test data obtained by the blood flow measurement unit and the motion sensing unit. Of course, in order to prevent the patient's overuse of analgesics, the maximum amount of drug that may be infused per hour may be limited according to a judgment of medical personnel, and the increased or decreased infusion amount may slowly return to the infusion amount prior to the occurrence of the condition if it deviates from the condition responsible for the change in infusion amount.

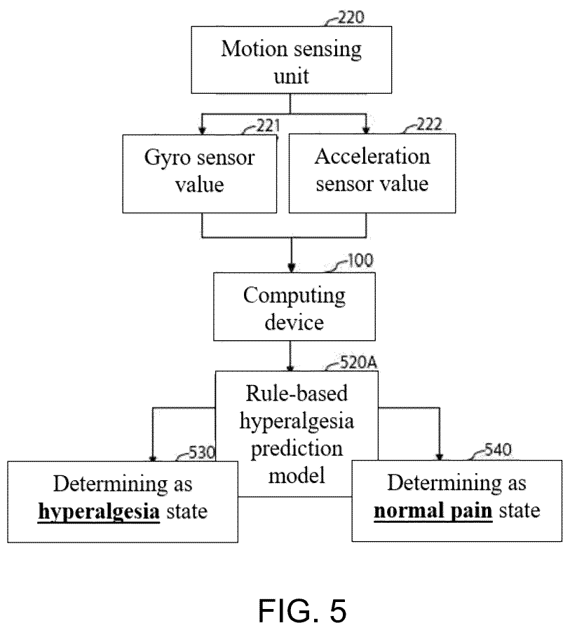

FIG. 5 is a flow chart showing a method of predicting the hyperalgesia state of the patient using a rule-based hyperalgesia prediction model, according to an embodiment of the present disclosure.

First, the rule-based model (rule-based hyperalgesia prediction model, or rule-based oversedation prediction model) mentioned in the present disclosure is a model that performs a logical operation according to a predetermined policy. For example, a rule-based model for classifying quadrilaterals could be a combination of logical operations such as “Does it have four interior angles?”, “Does it have four vertices?”, and “Does it have four sides?”.

In an embodiment, the processor 110 may receive the gyro sensor value 221 and the acceleration sensor value 222 from the motion sensing unit 220 related to the patient. The processor 110 may then determine whether or not the patient is in the hyperalgesia state (hyperalgesia 530 or normal pain 540) using the rule-based hyperalgesia prediction model 520A based on the gyro sensor value 221 and the acceleration sensor value 222.

The hyperalgesia state may be understood as a state in which the patient's pain is more than a certain level. However, the criteria for pain may be subjective from patient to patient. Accordingly, the processor 110 of the present disclosure may assess a patient's pain based on the patient's gestures, which may be said to be relatively objective. For example, the processor 110 may determine that the more violent the gesture of the patient (as the body movement increases), the more severe the pain. In addition, the processor 110 may determine that the pain is more severe when the patient's gestures are discontinuous and not continuous. In addition, the processor 110 may determine that the pain is severe when the posture of the patient is different from a posture in which the patient generally feels comfortable. At this time, the degree to which the gesture of the patient is violent may be calculated based on the magnitude of the movement amount (not shown) of the patient, and a numerical value for determining whether the gesture of the patients is continuous or discontinuous may be predicted as an average slope magnitude between each point when the magnitude of the movement amount (not shown) is graphed.

In an embodiment related to the rule-based hyperalgesia prediction model 520A, the processor 110 may calculate a movement amount (not shown) using the rule-based hyperalgesia prediction model 520A based on the gyro sensor value 221 and the acceleration sensor value 222. The movement amount (not shown) may correspond to a summed value (x+y) of a variance value x of the gyro sensor value continuously measured between the predetermined first time point and the second time point, and a variance value y of the acceleration sensor value continuously measured in between the first time points and the second time points. At this time, the first time point and the second time point may be respectively understood as a sensor measurement start time point and a sensor measurement end time point. Of course, in terms of accuracy, the sensor values are continuously measured, and the narrower the measured interval, the better. However, in order to efficiently use processing resources, the processor 110 may adjust an interval at which the motion sensing unit 220 collects data, and control a start time and an end time of collecting data. The rule-based hyperalgesia prediction model 520A may determine that a patient is in the hyperalgesia state if the patient's movement amount (not shown) exceeds a predetermined upper limit. And, when the patient is not in the hyperalgesia state, it may be determined that the patient is in a normal pain state.

In the previous embodiment, in order to calculate the patient's movement amount (not shown), the sum of the variance value x of the gyro sensor values and the variance value y of the acceleration sensor values was calculated. However, in such a case, a problem may arise in that one of the variance value x of the gyro sensor value or the variance value y of the acceleration sensor is considered to be one-to-one despite being a numerical value that should be considered more important in predicting the hyperalgesia state. As an embodiment related to this, when calculating the patient's movement amount (not shown), the processor 110 may multiply x and y by weights a and b, respectively, and sum them (i.e., ax+by). At this time, a and b may be values that sum to 1; however, they are not limited thereto and may be determined by various ways. Of course, in the calculation of the movement amount (not shown), if only the variance value x of the gyro sensor value is to be used, the calculation may be performed by setting a to 1 and b to 0.

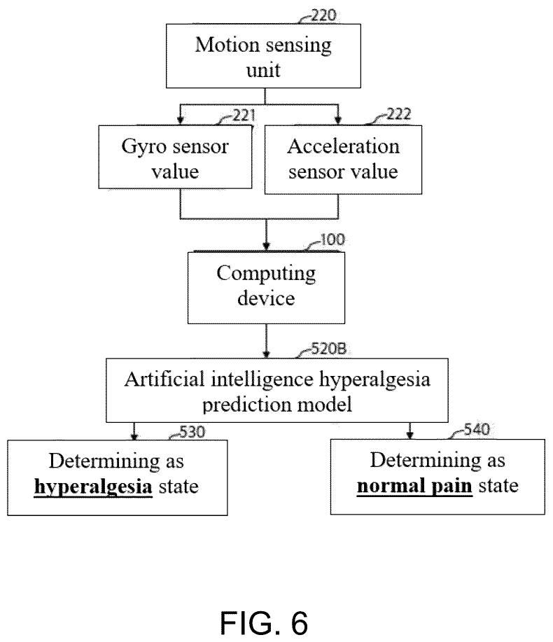

FIG. 6 is a flowchart showing a method of predicting the hyperalgesia state of the patient using an artificial intelligence hyperalgesia prediction model, according to an embodiment of the present disclosure.

The rule-based hyperalgesia prediction model 520A described above through FIG. 6 may be also replaced with the artificial intelligence hyperalgesia prediction model 520B. The artificial intelligence hyperalgesia prediction model may use a type of neural network model in the form of a neural network composed of nodes and edges. The artificial intelligence hyperalgesia prediction model 520B may perform a task of identifying an abnormal pattern by learning a pattern of gyro sensor values 221 (time series data_1) in a normal category, and a pattern of acceleration sensor values 222 (time series data_2) in a normal category based on learning data including sensing values measured from painless patients. In this case, the artificial intelligence hyperalgesia prediction model 520B may be a model that receives and encodes the time series data_1, and the time series data_2 as input data, decodes the encoded input data, and classifies the hyperalgesia state and the normal hyperalgesia state based on the decoded input data. However, the artificial intelligence hyperalgesia prediction model is not limited thereto, and various types of models such as auto-encoder type neural network model, SVM, SVDD, CNN, or LSTM may be used.

As an embodiment, the rule-based model 520A described in FIG. 5 and the artificial intelligence hyperalgesia prediction model 520B described in FIG. 6 may be used in combination to predict the hyperalgesia state of patient. For example, the processor 110 may predict the hyperalgesia state of the patient using the artificial intelligence hyperalgesia prediction model 520B only when the hyperalgesia state is predicted using the rule-based oversedation prediction model 520A. This embodiment may be said to be efficient in that it may use relatively little of the artificial intelligence hyperalgesia prediction model 520B, which consumes a lot of processing resources.

Alternatively, even if the artificial intelligence hyperalgesia prediction model 520B predicts that the patient is in normal pain, if the rule-based hyperalgesia predictive model 520A predicts a hyperalgesia state, the processor 110 may finally predict that the patient is hyperalgesia. This embodiment has an effect of correcting a prediction error of the artificial intelligence hyperalgesia prediction model 520B when the reliability of the artificial intelligence hyperalgesia prediction model 520B is low.

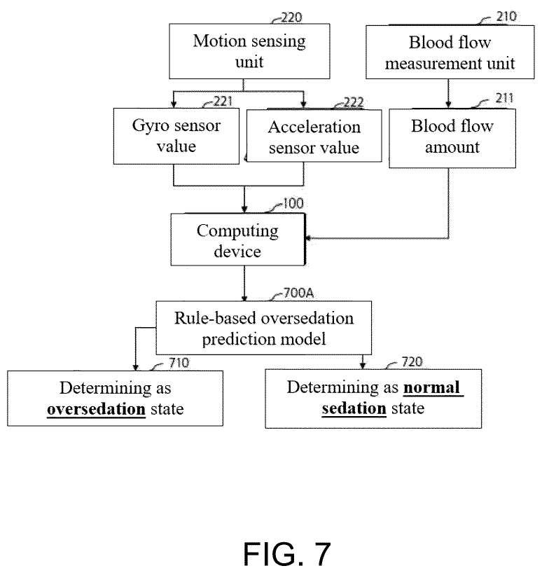

FIG. 7 is a flowchart showing a method of predicting the oversedation state of the patient using a rule-based oversedation prediction model, according to an embodiment of the present disclosure.

A method for the processor 110 to predict the oversedation state is described based on FIG. 7. Prior to the description, the oversedation state has a tendency to decrease the activity of the sympathetic nerve, increase the blood flow amount of the patient, and decrease the movement amount (not shown) as compared to the sedation state or the excitation state. The oversedation state is a state in which the patient does not easily awake due to drug, but the consciousness is lowered to the extent that the patient intentionally responds to repeated stimuli or pain stimuli. That is, since the patient's consciousness is decreased, the patient is relatively less sensitive to pain. Thus, patients in oversedation state require relatively lower amount of analgesics. In addition, when analgesics are overused in the patient in oversedation state, a side effect is likely to occur, and thus it is necessary to decrease infusion amount of the analgesic of the patient-controlled analgesia.

Referring now to FIG. 7, embodiments of how the processor 110 of the present disclosure predicts the oversedation state of patient using the rule-based oversedation prediction model are described.

First, the processor 110 of the computing device 100 may obtain the gyro sensor value 221 and the acceleration sensor value from the motion sensing unit that measures test data from the patient, and obtain the blood flow amount 211 from the blood flow measurement unit that measures the test data from the patient. Then, the processor 110 may calculate the movement amount based on the gyro sensor value and the acceleration sensor value (the method of calculating the movement amount may be the same as the aforementioned method of calculating the movement amount.) and may predict the oversedation state 710 or normal sedation state 720 based on the movement amount (not shown) and the blood flow amount 211 using the rule-based oversedation prediction model 700A. In this case, the rule-based oversedation prediction model 700A may predict a time point at which the movement amount (not shown) does not exceed a predetermined lower limit of movement amount, but the blood flow amount exceeds a predetermined upper limit of the blood flow amount, as the oversedation state. When the lower limit of movement amount is a1 and the upper limit of blood flow amount is b1, the said a1 and b1 may be determined according to the judgment of medical personnel. Alternatively, the said a1 and b1 may be replaced with a2 and b2, which are corrected values based on the patient's height, weight, blood pressure, and average heart rate by the processor 110, by using the default a1 and b1 determined according to the judgment of medical personnel.

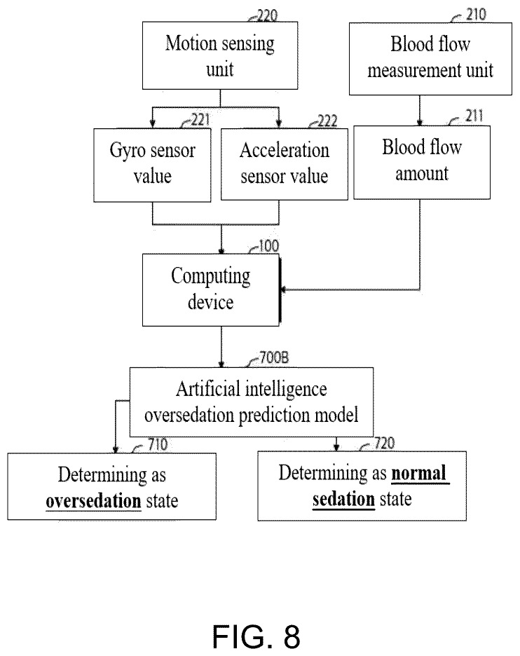

FIG. 8 is a flowchart showing a method of predicting the oversedation state of the patient using an artificial intelligence oversedation prediction model, according to an embodiment of the present disclosure.

In the foregoing, a method of predicting oversedation state of patient by using rule-based oversedation prediction model by the processor 110 has been described based on FIG. 7. In this case, the rule-based oversedation prediction model may be replaced with the artificial intelligence oversedation prediction model to predict the oversedation state of the patient. The artificial intelligence oversedation prediction model may use a type of neural network model in the form of a neural network composed of nodes and edges. The artificial intelligence oversedation prediction model 700B may perform a task of identifying an abnormal pattern by learning a pattern of gyro sensor values 221 (time series data_1) in a normal category, a pattern of acceleration sensor values 222 (time series data_2) in a normal category and a pattern of blood flow amount values 211 (time series data_3) in a normal category based on learning data including sensing values measured from painless patients. In this case, the artificial intelligence oversedation prediction model 700B may be a model that receives and encodes the time series data_1, the time series data_2, and the time series data_3 as input data, decodes the encoded input data, and classifies the oversedation state and the normal sedation state (state other than the oversedation state) based on the decoded input data. However, the artificial intelligence oversedation prediction model 700B is not limited thereto, and various types of models such as auto-encoder type neural network model, SVM, SVDD, CNN, or LSTM may be used.

The processor 110 may use a combination of the rule-based oversedation prediction model 700A and the artificial intelligence oversedation prediction model 700B to predict the oversedation state. This is to compensate for each other's disadvantages.

For example, the processor 110 may predict the oversedation state of the patient using the artificial intelligence hyperalgesia prediction model 700B only when the oversedation state is predicted using the rule-based oversedation prediction model 700A. This embodiment may be said to be efficient in that it may use relatively little of the artificial intelligence oversedation prediction model 700B, which consumes a lot of processing resources.

Alternatively, even if the artificial intelligence oversedation prediction model 700B predicts that the patient is in normal pain, if the rule-based oversedation predictive model 700A predicts an oversedation state, the processor 110 may finally predict that the patient is in an oversedation state. This embodiment has an effect of correcting a prediction error of the artificial intelligence oversedation prediction model 700B when the reliability of the artificial intelligence oversedation prediction model 700B is low.

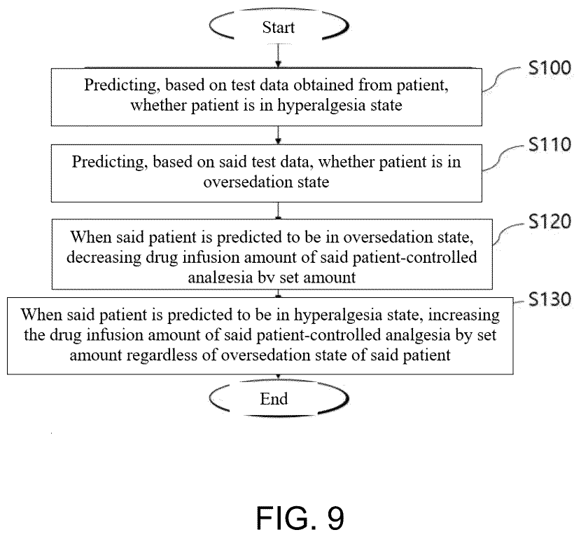

FIG. 9 is a flow chart representing a schematic flow for the method of controlling patient-controlled analgesia, according to an embodiment of the present disclosure.

Referring now to FIG. 9, a schematic flow for a method of controlling the patient-controlled analgesia, performed by at least one processor of computing device is described. The method may include predicting, by the processor 110, whether the patient is in a hyperalgesia state based on test data obtained from a patient (S100), predicting, by the processor 110, whether the patient is in a n oversedation state based on the test data (S110), decreasing, when the processor 110 predicts that the patient is in the oversedation state, a drug infusion amount of the patient-controlled analgesia by a set amount (S120), and increasing, when the patient is predicted to be in the hyperalgesia state, the drug infusion amount of the patient-controlled analgesia by a set amount regardless of the oversedation state of the patient (S130).

At this time, the method may further include predicting (S140, not shown), by the processor 110, a time point at which the hyperalgesia state button signal is received from the patient-controlled analgesia, as the hyperalgesia state, and the patient-controlled analgesia may include the hyperalgesia status button manually operable by the patient.

In addition, the test data may include blood flow amount measured using the blood flow measurement unit, and movement amount (not shown) measured using a motion sensing unit.

In addition, the motion sensing unit may include a gyro sensor and an acceleration sensor, and the movement amount (not shown) measured using the motion sensing unit may correspond to a summed value x+y of a variance value x of continuously measured gyro sensor value between a predetermined first time point and a second time point, and a variance value y of continuously measured acceleration sensor values between the first time point and the second time point.

In addition, the movement amount (not shown) measured using the motion sensing unit may correspond to summed value ax+by of the values ax, by, the values ax, by being obtained by weighting the x and the y with the predetermined weights a and b, respectively.

In addition, the predicting, by the processor 110, whether the patient is in hyperalgesia state may include predicting a time point at which the movement amount (not shown) exceeds a predetermined upper limit of the movement amount (not shown), as the hyperalgesia state.

In addition, the predicting, by the processor 110, whether the patient is in oversedation state may include predicting a time point at which the movement amount (not shown) does not exceed a predetermined lower limit of movement amount (not shown), but the blood flow amount exceeds a predetermined upper limit of the blood flow amount, as the oversedation state.

In the above, all the components constituting the embodiments of the present disclosure have been described as being combined as a single entity or operating in combination, but, the present disclosure is not necessarily limited to such embodiments. That is, within the scope of the disclosure, all components may also be optionally combined in one or more configurations to operate. In addition, the terms such as “include,” “comprise,” or “have” described above mean that the components may be inherent unless specifically stated to the contrary, and thus should be interpreted as not excluding other components but further including other components. All terms, including technical or scientific terms, have the same meaning as commonly understood by one of the ordinary skill in the art to which this disclosure belongs, unless otherwise defined. Generally used terms, such as predefined terms, should be construed as consistent with their meaning in the context of the relevant art and are not to be interpreted in an idealized or overly formal sense unless expressly so defined herein.

The above description is merely illustrative of the technical idea of the present disclosure, and various modifications and variations may be made by those skilled in the art without departing from the essential characteristics of the present disclosure. Therefore, the embodiments disclosed in the present disclosure are not intended to limit the technical idea of the present disclosure, but are intended to be illustrative, and the scope of the technical idea is not limited by these embodiments. The scope of protection of the present disclosure should be interpreted according to the following claims, and all technical ideas falling within the scope equivalent thereto should be interpreted as being included in the scope of rights of the present disclosure.

What is claimed is:

1. A method of controlling patient-controlled analgesia, performed by at least one processor of a computing device, the method comprising:
predicting, based on test data obtained from a patient, whether the patient is in a hyperalgesia state;
predicting, based on the test data, whether the patient is in an oversedation state;
decreasing, when the patient is predicted to be in the oversedation state, a drug infusion amount of the patient-controlled analgesia by a set amount; and
increasing, when the patient is predicted to be in the hyperalgesia state, the drug infusion amount of the patient-controlled analgesia by a set amount regardless of the oversedation state of the patient.

2. The method of controlling patient-controlled analgesia of claim 1, further comprising:
predicting a time point at which a hyperalgesia status button signal is received from the patient-controlled analgesia, as the hyperalgesia state;
wherein the patient-controlled analgesia comprises:
a hyperalgesia status button manually operable by the patient.

3. The method of controlling patient-controlled analgesia of claim 1, wherein the test data comprises:
a blood flow amount measured using the blood flow measurement unit; and
a movement amount measured using a motion sensing unit.

4. The method of controlling patient-controlled analgesia of claim 3, wherein the motion sensing unit comprises:
a gyro sensor and an acceleration sensor, and
wherein the movement amount (not shown) measured using the motion sensing unit corresponds to:
a summed value (x+y) of a variance value (x) of continuously measured gyro sensor value between a predetermined first time point and a second time point; and
a variance value (y) of continuously measured acceleration sensor values between the first time point and the second time point.

5. The method of controlling patient-controlled analgesia of claim 4, wherein the movement amount measured using the motion sensing unit corresponds to:
a summed value (ax+by) of the values (ax, by), the values (ax, by) being obtained by weighting the x and the y with the predetermined weights a and b, respectively.

6. The method of controlling patient-controlled analgesia of claim 5, wherein the predicting whether the patient is in hyperalgesia state comprises:
predicting a time point at which the movement amount (not shown) exceeds a predetermined upper limit of the movement amount, as the hyperalgesia state.

7. The method of controlling patient-controlled analgesia of claim 3, wherein the predicting whether the patient is in oversedation state comprises:
predicting a time point at which the movement amount (not shown) does not exceed a predetermined lower limit of movement amount, but the blood flow amount exceeds a predetermined upper limit of the blood flow amount, as the oversedation state.
